(19) United States
(12) Patent Application Publication
(10) Pub. No.: US 2021/0246159 A1
(43) Pub. Date: Aug. 12, 2021

(54) NOVEL FORM OF BARDOXOLONE METHYL

(71) Applicant: Johnson Matthey Public Limited Company, London (GB)

(72) Inventor: Nicholas PASCHALIDES, Devens, MA (US)

(73) Assignee: Johnson Matthey Public Limited Company, London (GB)

(21) Appl. No.: 17/248,816

(22) Filed: Feb. 9, 2021

Related U.S. Application Data
(60) Provisional application No. 62/972,302, filed on Feb. 10, 2020.

(51) Int. Cl. C07J 63/00; C07J 75/00

(52) U.S. Cl. CPC C07J 63/008; C07B 2200/13; C07J 75/00

(57) ABSTRACT

The present disclosure relates to a formic acid solvate of bardoxolone methyl and to a process for its preparation. The present disclosure also relates to pharmaceutical compositions comprising the formic acid solvate of bardoxolone methyl and to its use in a method for treating a disease.

### CROSS-REFERENCE TO RELATED APPLICATIONS

This application claims priority to U.S. Provisional Patent Application No. 62/972,302, filed Feb. 10, 2020, the disclosure of which is incorporated herein by reference in its entirety for all purposes.

### FIELD OF THE DISCLOSURE

The present disclosure relates to a formic acid solvate of bardoxolone methyl and to a process for its preparation. The present disclosure also relates to pharmaceutical compositions comprising the formic acid solvate of bardoxolone methyl and to its use in a method for treating a disease.

### BACKGROUND OF THE DISCLOSURE

Bardoxolone methyl, having the chemical designation, methyl (4aS,6aR,6bS,8aR,12aS,14aR,14bS)-11-cyano-2,2,6a,6b,9,9,12a-heptamethyl-10,14-dioxo-1,3,4,5,6,7,8,8a,14a,14b-decahydropicene-4a-carboxylate, is an experimental, oral, once-daily activator of Nrf2, a transcription factor that induces molecular pathways that promote the resolution of inflammation by restoring mitochondrial function, reducing oxidative stress, and inhibiting pro-inflammatory signaling. Bardoxolone methyl has the following structure:

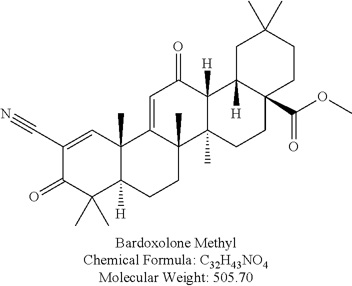

The FDA has granted Orphan Drug designation to bardoxolone methyl for the treatment of Alport syndrome, pulmonary arterial hypertension, and autosomal dominant polycystic kidney disease (ADPKD). The European Commission has granted Orphan Drug designation in Europe to bardoxolone methyl for the treatment of Alport syndrome. Bardoxolone methyl has been or is being studied in various clinical trials, including CARDINAL, a Phase 3 study for the treatment of Alport syndrome, CATALYST, a Phase 3 study for the treatment of connective tissue disease-associated pulmonary arterial hypertension, and AYAME, a Phase 3 study for the treatment of diabetic kidney disease in Japan.

There are several patents and patent application publications which disclose various solid state forms of bardoxolone methyl. For example, U.S. Pat. Nos. 8,309,601, 8,633,243, and 8,088,824 disclose an anhydrous form of bardoxolone methyl, amorphous bardoxolone methyl, a bardoxolone methyl dimethanol solvate, and a bardoxolone methyl hemibenzenate. WO2019/014412 discloses Form C, Form D, and From E of bardoxolone methyl wherein Form C has an X-ray powder diffraction pattern having peaks at 6.2, 12.4, 15.4, 18.6 and 24.9° 2θ±0.2° 2θ; Form D has an X-ray powder diffraction pattern having peaks at 3.6, 7.1, 10.8, 12.4 and 16.5° 2θ±0.2° 2θ; and Form E has an X-ray powder diffraction pattern having peaks at 7.0, 13.2, 13.9, 15.1 and 16.8° 2θ±0.2° 2θ. CN102887936 discloses a methylbenzene solvate, a hemi-dioxane solvate and a hemi-tetrahydrofuran solvate of bardoxolone methyl. CN102875634 discloses a bardoxolone methyl mono methanol solvate, an anhydrous form of bardoxolone methyl, and a dihydrate of bardoxolone methyl. None of these references disclose a formic acid solvate of bardoxolone methyl; more particularly a di-formic acid solvate.

### SUMMARY OF THE DISCLOSURE

The present invention is directed to a crystalline form of bardoxolone methyl as a formic acid solvate; more particularly a di-formic acid solvate of bardoxolone methyl, designated herein as Form A. The present invention is further directed to processes for the preparation of a formic acid solvate of bardoxolone methyl; more particularly, Form A. The present invention also is directed to pharmaceutical compositions comprising a formic acid solvate of bardoxolone methyl; more particularly, Form A, and to its use in a method for treating disease.

### BRIEF DESCRIPTION OF THE DRAWINGS

FIG. 1 provides a representative XRPD pattern of Form A.

FIG. 2 provides a three-dimensional structure of Form A that is discerned from SCXRD.

FIG. 3 provides a representative DSC plot of Form A.

FIG. 4 provides a representative isothermal DVS plot of Form A.

FIG. 5 provides a representative kinetic DVS plot of Form A.

FIG. 6 provides a representative TGA plot of Form A.

### DETAILED DESCRIPTION OF THE DISCLOSURE

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles described herein may be applied to other examples and applications without departing from the spirit and scope of the various embodiments. Therefore, the various embodiments are not intended to be limited to the examples described herein and shown but are to be accorded the scope consistent with the claims.

### Terms/Definitions

As used herein and unless otherwise specified, the terms “about” and “approximately,” when used in connection with a numeric value or a range of values which is provided to characterize a particular solid form, e.g., a specific temperature or temperature range, such as, e.g., that describing a DSC or TGA thermal event, including, e.g., melting, dehydration, desolvation or glass transition events; a mass change, such as, e.g., a mass change as a function of temperature or humidity; a solvent or water content, in terms of, e.g., mass or a percentage; or a peak position, such as, e.g., in analysis by IR or Raman spectroscopy or XRPD; indicate that the value or range of values may deviate to an extent deemed reasonable to one of ordinary skill in the art while still describing the particular solid form.

As used herein and unless otherwise specified, the term “pharmaceutical composition” is intended to encompass a pharmaceutically effective amount of a formic acid solvate of bardoxolone methyl; more particularly, Form A, and a pharmaceutically acceptable excipient. As used herein, the term “pharmaceutical compositions” includes pharmaceutical compositions such as tablets, pills, powders, liquids, suspensions, emulsions, granules, capsules, suppositories, or injection preparations.

As used herein and unless otherwise specified, the term “crystalline” and related terms used herein, when used to describe a compound, substance, modification, material, component or product, unless otherwise specified, mean that the compound, substance, modification, material, component or product is substantially crystalline as determined by X-ray diffraction. See, e.g., Remington: The Science and Practice of Pharmacy, 21st edition, Lippincott, Williams and Wilkins, Baltimore, Md. (2005); The United States Pharmacopeia, 23rd ed., 1843-1844 (1995).

As used herein and unless otherwise specified, the term “excipient” refers to a pharmaceutically acceptable organic or inorganic carrier substance. Excipients may be natural or synthetic substances formulated alongside the active ingredient of a medication, included for the purpose of bulking-up formulations that contain potent active ingredients (thus often referred to as “bulking agents,” “fillers,” or “diluents”), or to confer a therapeutic enhancement on the active ingredient in the final dosage form, such as facilitating drug absorption or solubility. Excipients can also be useful in the manufacturing process, to aid in the handling of the active substance, such as by facilitating powder flowability or non-stick properties, in addition to aiding in vitro stability such as prevention of denaturation over the expected shelf life.

As used herein and unless otherwise specified, the term “patient” refers to an animal, preferably a mammal, most preferably a human, who has been the object of treatment, observation or experiment. Preferably, the patient has experienced and/or exhibited at least one symptom of the disease or disorder to be treated and/or prevented. Further, a patient may not have exhibited any symptoms of the disorder, disease or condition to be treated and/or prevented, but has been deemed by a physician, clinician or other medical professional to be at risk for developing said disorder, disease or condition.

As used herein and unless otherwise specified, the terms “polymorph,” “polymorphic form” or related term herein, refer to a crystalline form of an API (active pharmaceutical ingredient) free base or salt thereof that can exist in two or more forms, as a result of different arrangements or conformations of the molecule, ions of the salt, or addition and arrangement of solvents or coformers within the crystalline lattice of the crystal form.

As used herein and unless otherwise specified, the terms “substantially” or “substantially free/pure” with respect to a crystalline form means that the form contains about less than 30 percent, about less than 20 percent, about less than 15 percent, about less than 10 percent, about less than 5 percent, or about less than 1 percent by weight of impurities. Impurities may, for example, include other polymorphic forms, water and solvents other than that in a designated polymorphic form.

As used herein and unless otherwise specified, the terms “treat,” “treating” and “treatment” refer to the eradication or amelioration of a disease or disorder, or of one or more symptoms associated with the disease or disorder. In certain embodiments, the terms refer to minimizing the spread or worsening of the disease or disorder resulting from the administration of one or more therapeutic agents to a patient with such a disease or disorder. In some embodiments, the terms refer to the administration of a compound provided herein, with or without other additional active agents, after the onset of symptoms of the particular disease. Bardoxolone methyl is intended to treat a patient with one or more of Alport syndrome, pulmonary arterial hypertension, autosomal dominant polycystic kidney disease (ADPKD), diabetic kidney disease, connective tissue disease-associated pulmonary arterial hypertension, and other forms of kidney disease.

As used herein and unless otherwise specified, the term “ambient temperature” refers to the working laboratory temperature range, about 18° C. to about 25° C.

As used herein and unless otherwise specified, the term “atmospheric pressure” refers to about 760 mm Hg.

It is therefore an object of the present disclosure to provide a formic acid solvate of bardoxolone methyl; more particularly, Form A, that is substantially pure, stable and scalable. It is also an object of the present disclosure to provide a formic acid solvate of bardoxolone methyl; more particularly, Form A, that is capable of being isolated and handled.

Techniques for characterizing crystal and amorphous forms include but are not limited to differential scanning calorimetry (DSC), thermal gravimetric analysis (TGA), dynamic vapor sorption (DVS), X-ray powder diffractometry (XRPD), single crystal X-ray diffraction (SCXRD), proton nuclear magnetic resonance (¹H-NMR), Fourier transform infrared spectroscopy (FTIR Spectroscopy), and Optical Microscopy.

TGA data are collected using a TA Instruments TGA Q500. Samples (about 5-10 mg) are placed in a hermetically sealed pre-tared aluminum sample pan with a pinhole placed in it and scanned from about 25 to about 350° C. at a rate of about 10° C./min using a nitrogen purge at about 60 mL/min.

XRPD patterns are obtained using a Bruker D8 Advance equipped with a Cu Kα radiation source (λ=1.54 Å), a 9-position sample holder and a LYNXEYE super speed detector. Samples are placed on zero-background, silicon plate holders for analysis. One skilled in the art would recognize that the ° 2θ values and the relative intensity values are generated by performing a peak search on the measured data and the d-spacing values are calculated by the instrument from the ° 2θ values using Bragg's equation. One skilled in the art would further recognize that the relative intensity for the measured peaks may vary as a result of sample preparation, orientation and instrument used, for example.

X-ray intensity data for SCXRD are collected on a Bruker D8QUEST [1] CMOS area detector employing graphite-monochromated Mo—Kα radiation (λ=0.71073 Å) at a temperature of about 100K.

DVS samples (about 5-10 mg) are analyzed using a TA Instruments Q5000 SA powerful dynamic vapor sorption analyzer. The relative humidity is started at about 50% and adjusted to between about 0-90% humidity with about a 10% increase or decrease in humidity for each step for 3 cycles. The weight of the sample is continuously monitored and recorded.

DSC data are collected using a TA Instruments Q10 DSC. Samples (about 2-8 mg) are placed in hermetically sealed aluminum sample pans and scanned from about 25 to about 350° C. at a rate of about 10° C./min under a nitrogen purge of about 50 mL/min.

In one embodiment, a formic acid solvate of bardoxolone methyl is prepared comprising:
a) dissolving bardoxolone methyl in formic acid to form a solution; and
b) allowing the formic acid solvate of bardoxolone methyl to precipitate from the solution.
   In one embodiment, the formic acid solvate of bardoxolone methyl is Form A. The ratio of bardoxolone methyl to formic acid is not critical as long as the bardoxolone methyl is substantially or completely dissolved in the formic acid. In one embodiment, the weight to volume ratio of bardoxolone methyl to formic acid is about 70 mg/mL-200 mg/mL. In one embodiment, the dissolving occurs at ambient temperature. The amount of time necessary for crystals to begin to precipitate can vary with temperature and the reaction scale. In one embodiment, crystals begin to precipitate in about 10 minutes. In another embodiment, crystals begin to precipitate in less than about 10 minutes. In other embodiments, crystals begin to precipitate in more than 10 minutes. In one embodiment, the crystals are removed from the solution after precipitation. In one embodiment, the solution is centrifuged and decanted to isolate the crystals from the solution. In one embodiment, the solution is then subjected to fast evaporation. In one embodiment, the fast evaporation comprises vacuum drying the solution at about −30 mm Hg and about 50° C. to yield a crystalline formic acid solvate of bardoxolone methyl; more particularly, Form A. In another embodiment, the solution is subjected to slow evaporation. In one embodiment, the slow evaporation comprises exposing the solution to atmospheric pressure and ambient temperature to yield a crystalline formic acid solvate of bardoxolone methyl; more particularly, Form A. In another embodiment, the solution is subjected to both fast evaporation and slow evaporation.

In another embodiment, a formic acid solvate of bardoxolone methyl is prepared comprising:
a) dissolving bardoxolone methyl in formic acid to form a solution; and
b) subjecting the solution to fast evaporation, slow evaporation, or a combination of fast and slow evaporation resulting in the formic acid solvate of bardoxolone methyl.
   In one embodiment, the formic acid solvate of bardoxolone methyl is Form A. As indicated above, the ratio of bardoxolone methyl to formic acid is not critical as long as the bardoxolone methyl is substantially or completely dissolved in the formic acid. In one embodiment, the weight to volume ratio of bardoxolone methyl to formic acid is about 70 mg/mL-200 mg/mL. In one embodiment, the dissolving occurs at ambient temperature. In one embodiment, the solution is subjected to fast evaporation. In one embodiment, the fast evaporation comprises vacuum drying the solution at about −30 mm Hg and about 50° C. In another embodiment, the solution is subjected to slow evaporation. In one embodiment, the slow evaporation comprises exposing the solution to atmospheric pressure and ambient temperature. In one embodiment, the solution is subjected to both fast evaporation and slow evaporation for a period of time.

The crystalline formic acid solvate of bardoxolone methyl; more particularly, Form A, can be dried by any conventional methods known to one of ordinary skill in the art. In one embodiment, it is desirable to avoid extensive drying of the crystalline formic acid solvate of bardoxolone methyl; more particularly, Form A, to avoid the potential for desolvation. In one embodiment, it is desirable to store the crystalline formic acid solvate of bardoxolone methyl; more particularly, Form A, away from high heat and humidity. Form A remains stable at 30° C. at 65% RH for at least 5 days in a capped vial.

An embodiment of the invention is directed to Form A that is a di-formic acid solvate. A further embodiment of the invention is Form A prepared by a process embodiment as described herein.

The present disclosure also encompasses a pharmaceutical composition comprising a formic acid solvate of bardoxolone methyl; more particularly, Form A, and a pharmaceutically acceptable excipient. The pharmaceutical composition may be prepared according to U.S. Pat. Nos. 8,747,901, 8,088,824 and 9,155,721, or any other methods known in the art.

The present disclosure provides for a method of treating disease by administering to a patient in need thereof, a pharmaceutical composition comprising a formic acid solvate of bardoxolone methyl; more particularly, Form A. Bardoxolone methyl is intended for the treatment of a patient with one or more of Alport syndrome, pulmonary arterial hypertension, autosomal dominant polycystic kidney disease (ADPKD), diabetic kidney disease, connective tissue disease-associated pulmonary arterial hypertension, and other forms of kidney disease.

The dosage of the pharmaceutical compositions may be varied over a wide range. Optimal dosages and dosage regimens to be administered may be readily determined by those skilled in the art, and will vary with the mode of administration, the strength of the preparation and the advancement of the disease condition. In addition, factors associated with the particular patient being treated, including patient's sex, age, weight, diet, physical activity, time of administration and concomitant diseases, will result in the need to adjust dosages and/or regimens.

### EXAMPLES

Examples 1-3, which follow herein, provide embodiments of the preparation of a formic acid solvate of bardoxolone methyl, more particularly, Form A.

The Examples are presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles described herein may be applied to other examples and applications without departing from the spirit and scope of the various embodiments. Therefore, the various embodiments are illustrative of the present disclosure and the disclosure is not intended to be limited to the examples described herein and shown.

### Example 1

### Preparation of Form A

About 200 mg of bardoxolone methyl is dissolved in about in 1 mL of formic acid at ambient temperature. Precipitation occurs after about 10 minutes. These crystals are identified as Form A.

Form A is characterized by its XRPD pattern peaks. 20 and relative % intensity values for peaks are shown in Table I.

TABLE 1
Average Peak List for
Form A diffractogram

| Angle (2θ) | |
|---|---|
| degree | Intensity % |
| 4.2 | 48.2 |
| 8.2 | 100.0 |
| 8.9 | 56.3 |
| 11.4 | 68.5 |
| 12.3 | 59.5 |
| 13.8 | 52.3 |
| 15.1 | 44.6 |
| 15.9 | 44.1 |
| 16.4 | 45.9 |
| 16.7 | 51.4 |
| 17.0 | 58.3 |
| 17.8 | 34.7 |
| 18.2 | 34.2 |
| 18.6 | 35.8 |
| 20.1 | 41.0 |
| 20.5 | 35.4 |
| 21.0 | 34.5 |
| 24.7 | 58.8 |
| 31.5 | 34.5 |
| 33.1 | 36.3 |

The angle measurements are ±0.2° 2Θ. In one embodiment, key defining peaks for Form A include two or more of 8.2, 11.4, 12.3, 17.0, and 24.7° 2Θ. In another embodiment, key defining peaks for Form A further comprise one or more peaks selected from 8.9, 13.8, and 16.7° 2Θ.

Single crystal data for Form A confirms that it is a di-formic acid solvate. Single crystal parameters for Form A as determined by SCXRD are:

a=6.4 Å±1.5%
b=11.2 Å±1.5%
c=21.5 Å±1.5%
α=γ=90°
β=97°±3°

### Cell Volume=1541 Å³±3%

Crystal system: monoclinic space group

### Space Group: P2₁

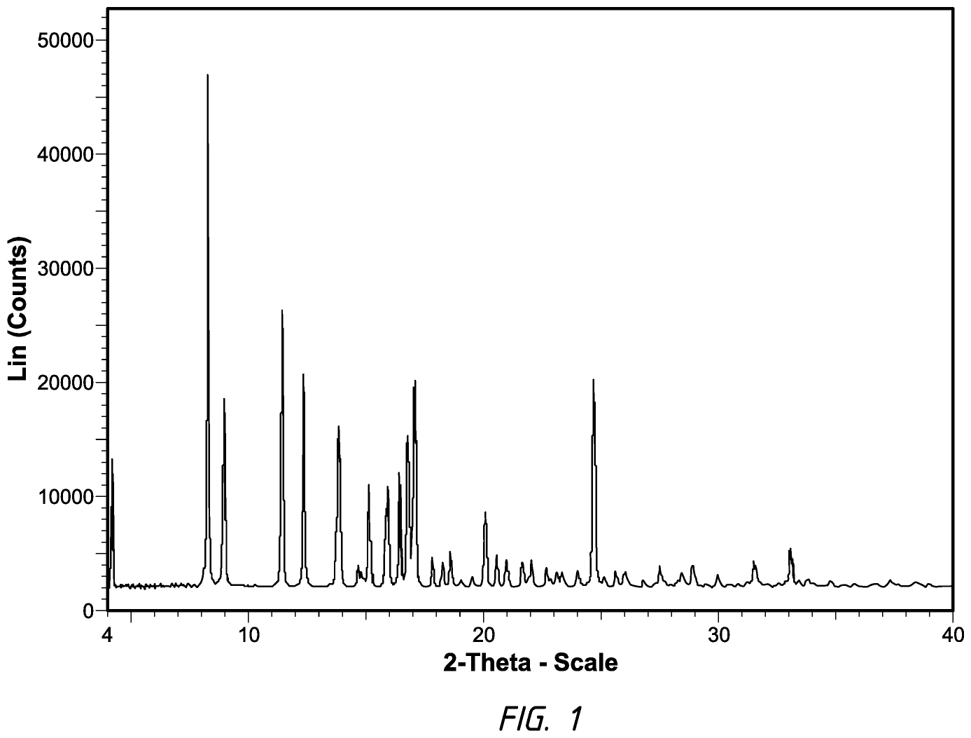

FIG. 1 provides a representative XRPD pattern for a representative sample of Form A.

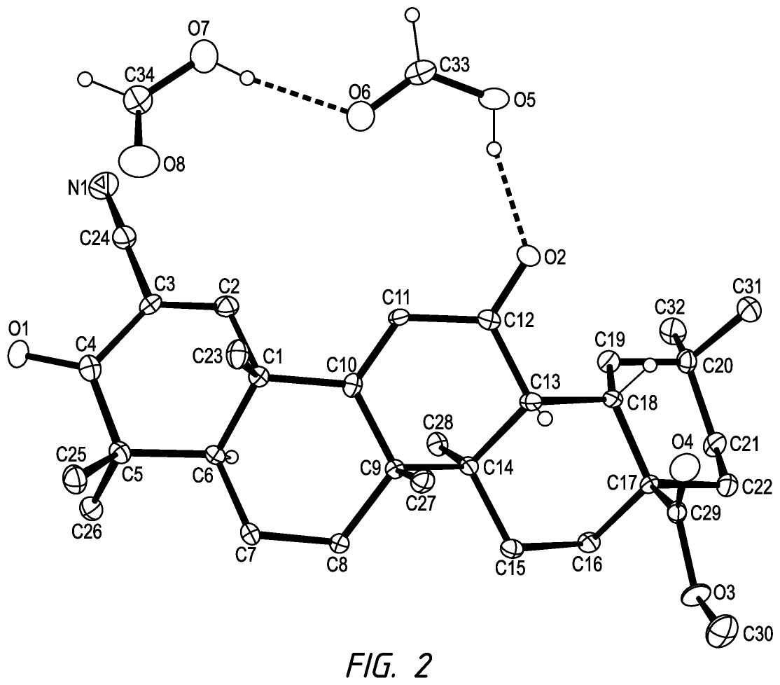

FIG. 2 provides a three-dimensional structure of Form A that is discerned from SCXRD.

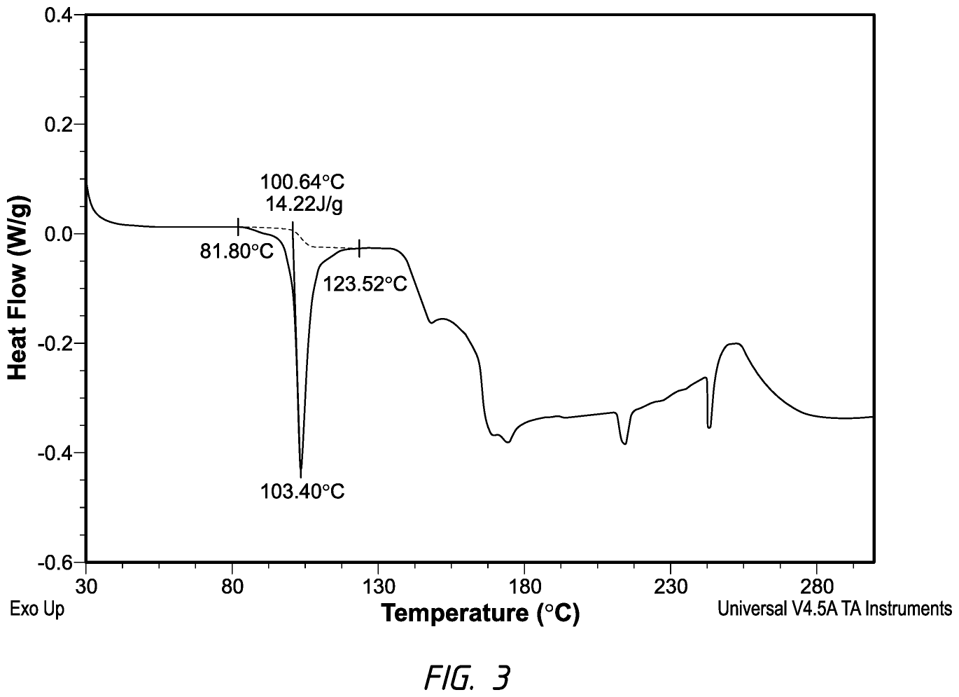

FIG. 3 provides a representative DSC plot of Form A which shows the onset of an endothermic event at about 101° C.

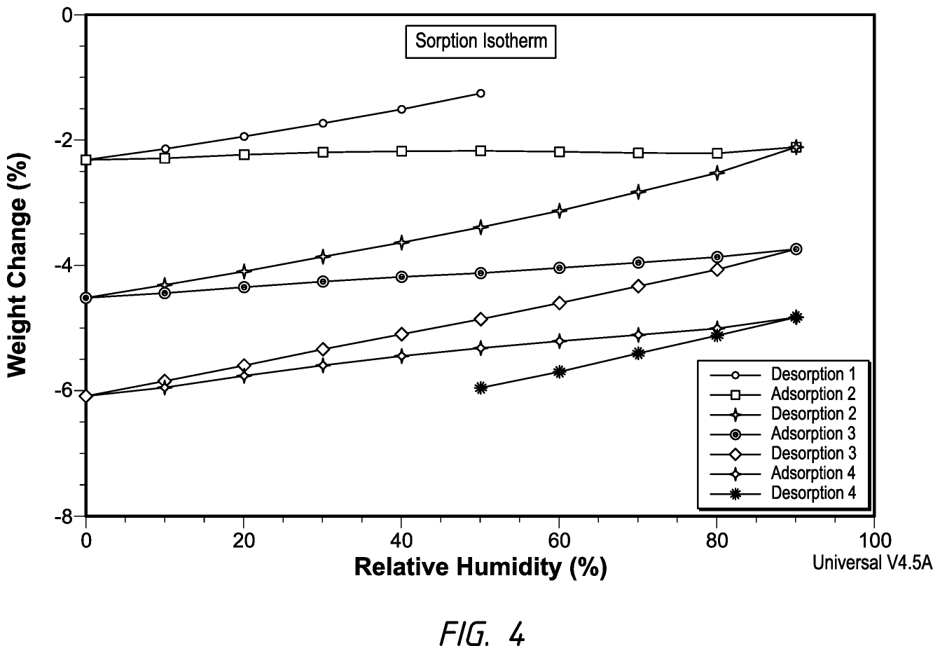

FIG. 4 provides a representative isothermal DVS plot of Form A.

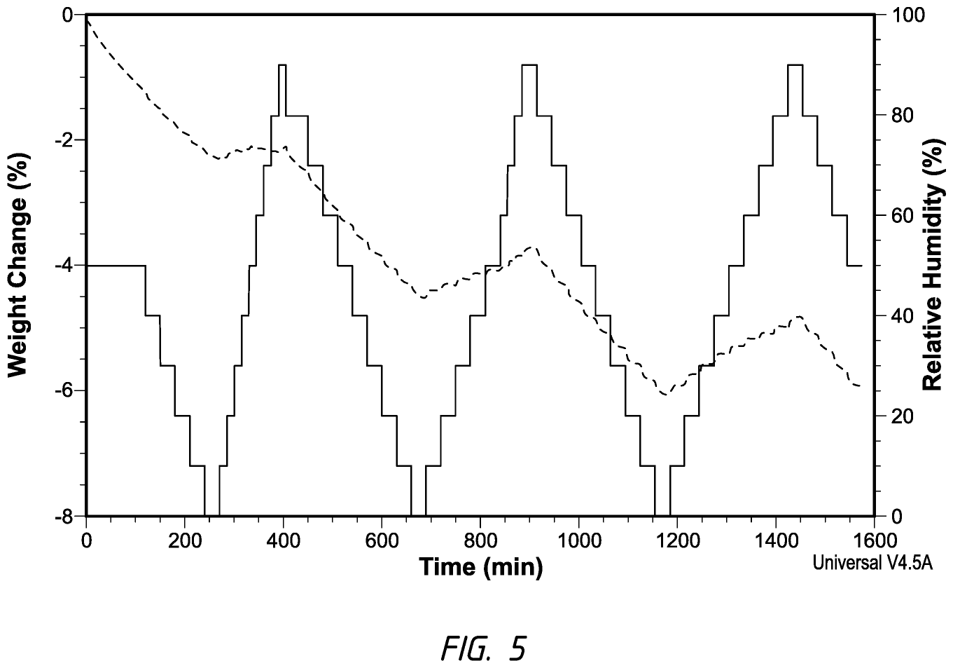

FIG. 5 provides a representative kinetic DVS plot of Form A that shows a degree of solvation when Form A is exposed to from 0 to 90% RH.

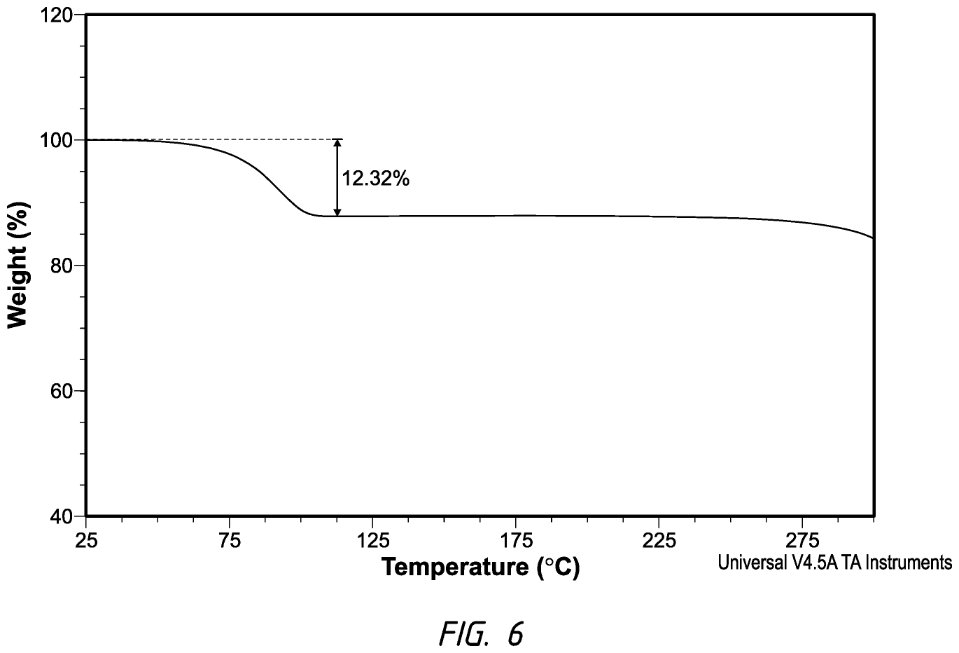

FIG. 6 provides a representative TGA plot of Form A which shows about a 12% weight loss. It is unclear if there is degradation or conversion to another form after 120° C.

### Examples 2a and 2b

### Preparation of Form A

About 200 mg of bardoxolone methyl is dissolved in about 1 mL of formic acid at ambient temperature. Precipitation occurs after about 10 minutes. The solution is centrifuged and the clear supernatant is decanted.

### Example 2a

The supernatant is vacuum dried at about −30 mm Hg and about 50° C. for a few hours until it dries resulting in Form A.

### Example 2b

The supernatant is placed in an uncapped vial at atmospheric pressure and ambient temperature to dry. Large crystals of Form A are generated over time.

### Example 3

### Preparation of Form A—5 g Scale

| Chemicals/ | | Mol. | No. of | | |
|---|---|---|---|---|---|
| Reagents | Amount | Weight | mMoles | Equival. | Density |
| Bardoxolone | 5 g | 505.70 | 9.887 | 1 mass (g) | N/A |
| Methyl | | | | | |
| Formic Acid | 20 mL | 46.03 | 530 | 4 vol (mL) | 1.22 |
| | (24.4 g) | | | | |

A 40 mL scintillation vial is fitted with a magnetic stirring bar and a rubber septum cover and used as a reactor vessel. Through the septum, a J-Chem temperature probe is inserted to monitor the reactor temperature. A needle is also inserted into the septum to prevent pressure build-up. The vial is placed on a hot plate. Bardoxolone methyl (5 g, 9.9 mmoles) is charged into the reactor with the hot plate set to 50° C.

Formic acid is charged in one-volume to mass increments (5 mL each time) while heating the mixture. The slurry is very thick after addition of 5 and 10 mL of formic acid. After addition of 20 mL (4 volumes) of formic acid and a slurry temperature of 42° C., a clear solution is obtained.

Precipitation occurs after cooling slowly to ambient temperature by turning off hot plate and then by faster cooling down to about 5° C. using a “Polar Bear” heating/cooling unit.

The mixture is vacuum filtered (−17.5 mm Hg) using a Buchner funnel. The funnel is fitted with PEPCAP polyester cloth (5 μL pore size). The amount of solids remaining on the reactor's walls is negligible, however, the cooled filtrate can be fed back into the reactor to transfer any leftover solids from the vessel if needed.

The solids are dried in a vacuum oven at about −29 mm Hg at about 42° C. for about 3 hours resulting in about 4.4 g of solids. The solids are analyzed by XRPD. The oven temperature is reduced to about 30° C. and the solids are dried under vacuum at about −29 mm Hg overnight (about 17 hours) resulting in about 4.34 g of drier solids (about 73.4% isolated yield). The solids are analyzed by XRPD and determined to be Form A.

The above examples are set forth to aid in the understanding of the disclosure and are not intended and should not be construed to limit in any way the disclosure set forth in the claims which follow hereafter.

1. Formic acid solvate of bardoxolone methyl.

2. The formic acid solvate of bardoxolone methyl of claim 1, which is a di-formic acid solvate, wherein the di-formic acid solvate is Form A.

3. The formic acid solvate of bardoxolone methyl of claim 2, which is characterized by having at least 2 or more X-ray powder diffraction peaks selected from about 8.2, 11.4, 12.3, 17.0, and 24.7° 2Θ±0.2° 2Θ as measured by CuKα radiation.

4. The formic acid solvate of bardoxolone methyl of claim 3, further comprising one or more X-ray powder diffraction peaks selected from about 8.9, 13.8, and 16.7° 2Θ±0.2° 2Θ as measured by CuKα radiation.

5. The formic acid solvate of bardoxolone methyl of claim 2, which is characterized by an onset of an endothermic event at about 101° C.±3° C., as measured by differential scanning calorimetry.

6. A process for the preparation of the formic acid solvate of bardoxolone methyl of claim 1 comprising:
a) dissolving bardoxolone methyl in formic acid to form a solution; and
b) allowing the formic acid solvate of bardoxolone methyl to precipitate from the solution.

7. The process of claim 6, wherein the formic acid solvate of bardoxolone methyl is Form A.

8. The process of claim 6, wherein the weight to volume ratio of bardoxolone methyl to formic acid is about 70 mg/mL to about 200 mg/mL.

9. The process of claim 6, wherein the dissolving occurs at ambient temperature.

10. The process of claim 6, further comprising:
c) separating the formic acid solvate of bardoxolone methyl precipitate from the solution; and
d) subjecting the solution to fast evaporation, slow evaporation, or a combination of fast and slow evaporation resulting in the formic acid solvate of bardoxolone methyl.

11. The process of claim 10, wherein the formic acid solvate of bardoxolone methyl of step d) is Form A.

12. A process for the preparation of the formic acid solvate of bardoxolone methyl of claim 1 comprising:
a) dissolving bardoxolone methyl in formic acid to form a solution; and
b) subjecting the solution to fast evaporation, slow evaporation, or a combination of fast and slow evaporation resulting in the formic acid solvate of bardoxolone methyl.

13. The process of claim 12, wherein the formic acid solvate of bardoxolone methyl is Form A.

14. The process of claim 12, wherein the weight to volume ratio of bardoxolone methyl to formic acid is about 70 mg/mL to about 200 mg/mL.

15. The process of claim 12, wherein the dissolving occurs at ambient temperature.

16. The process of claim 12, wherein the fast evaporation comprises vacuum drying the solution at about −30 mm Hg and about 50° C.

17. The process of claim 12, wherein the slow evaporation comprises exposing the solution to atmospheric pressure and ambient temperature.

18. The formic acid solvate of bardoxolone methyl of claim 2, which has single crystal parameters:
a=6.4 Å±1.5%
b=11.2 Å±1.5%
c=21.5 Å±1.5%
α=γ=90°
β=97°±3°

19. The formic acid solvate of bardoxolone methyl of claim 2, which has a cell volume of about 1541 Å³±3%.

20. The formic acid solvate of bardoxolone methyl of claim 2, which has a monoclinic space group.

21. The formic acid solvate of bardoxolone methyl of claim 18, which has a P2₁ space group.
